(19) United States
(12) Patent Application Publication
(10) Pub. No.: US 2022/0214340 A1
(43) Pub. Date: Jul. 7, 2022

(54) ZIPPER STRUCTURE THAT HELPS THE FORMATION OF PROTEIN DIMER AND APPLICATION THEREOF

(71) Applicant: LEIDE BIOSCIENCES CO., LTD., Guangzhou (CN)

(72) Inventors: Xiang YANG, Guangzhou (CN); Jianrong LOU, Guangzhou (CN); Guihua XIE, Guangzhou (CN)

(21) Appl. No.: 17/607,007

(22) PCT Filed: Apr. 26, 2020
(86) PCT No.: PCT/CN2020/086975

§ 371 Date: Oct. 27, 2021

(30) Foreign Application Priority Data
Apr. 28, 2019 (CN) 2019 10348168.4

(51) Int. Cl. G01N 33/564; C07K 7/50; C12N 15/62; G01N 33/68

(52) U.S. Cl. CPC G01N 33/564; G01N 33/68; C12N 15/62; C07K 7/50

(57) ABSTRACT

The present invention relates to the field of genetic engineering, and provides a zipper fastener structure of promoting formation of a protein dimer and application thereof. The zipper fastener can be applied to dimerization of proteins of the same type and dimerization of proteins of different types, and can also be applied to polypeptide cycle formation, polypeptide dimerization, and polypeptide extension. A ESAT6-CFP 10 dimer having an approximately native conformation can be obtained, and the dimer has better solubility, and has a better stimulating effect on memory T cells than a ESAT6-CFP10 fusion protein capable of linear fusion expression. A dimer zipper fastener can assist the formation of a more stable cyclic polypeptide, and a CCP polypeptide added with a dimer fastener can improve the detection rate for citrullinated autoantibodies in serum of a rheumatoid arthritis patient.

### TECHNICAL FIELD

The present disclosure relates to the field of biology, in particular to the field of protein. More particularly, the present disclosure relates to a zipper fastener structure promoting formation of a protein dimer and use thereof.

### BACKGROUND

A protein dimer is a quaternary structure of the protein. A homodimer is composed of two same protein molecules (the process for forming the homodimer is called homodimerization). A heterodimer is composed of two different kinds of protein molecules (the process for forming the heterodimer is called heterodimerization). In the biochemical field, most of the dimers are not formed via covalent linkages. For example, reverse transcriptase is a heterodimer enzyme formed with two kinds of different amino acid chains that are bonded via non-covalent linkages. Another example is protein dimer NEMO, which is a dimer formed via a disulfide bond. Some proteins include a special region that ensures dimerization (dimerization region). Dimerization of proteins plays an important role in the growth, reproduction, signaling of cells. The study of protein dimers is important to understanding of functions, production and uses of proteins. It is a challenging task to design dimers of proteins.

Currently, there are some methods for designing protein dimers. Cysteine knot with more than three cysteines was designed at C-terminus of the target protein for dimer formation (Sherilyn L, 2001). A protein dimer prepared by this method has high affinity, but poor stability with easy polymerization and precipitation. Knob-in-hole structure is designed by modification of Fc fragment of an antibody, and has been used in developing bispecific antibodies (Elliot J M. 2014). Leucine zipper is a structural motif occurring in DNA binding protein and other proteins, in which protein sequences, leucine regularly appears at every 7th amino acid position. An a-helix of the protein has 3.6 amino acid residues per turn. When the primary structure forms an α-helix, leucine residues are bonded to line up parallel to the helix axis on the same line on the outside, and occur once every two turns. Two sets of parallel-oriented α-helices with leucine form a symmetric dimer. The leucine residues on the dimer and the branched carbon chain on the side chains are staggered with each other, hence it is called leucine zipper. This structure has an excessively large portion of the fusion protein and is not suitable as a dimer fusion protein. Disulfide bonds are often used in the design of protein dimers, but often cause problems that outweigh the benefits. Many proteins have cysteine in themselves, and these cysteine residues play a significant role in the three-dimensional structure and function of the protein. When the protein is expressed, the redundant disulfide bonds will interfere with the folding of the recombinant protein, resulting in the formation of protein inclusion bodies. And excess cysteine can also cause multimer formation.

The three-dimensional structure of a native protein is very complex. In native proteins, besides interaction between ionic bonds, hydrogen bonds and interaction between hydrophobic amino acids dominate. Complementation of the native three-dimensional structures are more important for two proteins to form a stable dimer, which increased the difficulty of designing the recombinant protein dimers.

FLAG-tag is a conventional tag for testing protein expression, and is often used for purifying recombinant proteins. FLAG-tag has an amino acid sequence of DYKDDDDK. His-tag is a fusion tag composed of six histidine residues, and can be added to C-terminus or N-terminus of a target protein. When the tag is used, on one hand, it can form an epitope that facilitates purification and detection; on the other hand, a unique structural feature (a binding ligand) can be generated to facilitate purification. There is a strong attraction between the side chain of the histidine residue and solid nickel. Therefore, immobilized metal-chelate affinity chromatography (IMAC) filler can be used in affinity separation and purification of recombinant protein with His-tag. The other tags may comprise V5-tag with a sequence of GKPIPNPLLGLDST; S-tag with a sequence of KETAAAKFERQHMDS; Myc-tag with a sequence of EQKLISEEDL; HA-tag with a sequence of YPYDVPDYA; and MBP-tag, and the like.

5′-terminus of some genes has a relatively short RNA sequence (ranging from 150 bp to 250 bp in length). Such RNA sequences can be folded to form an initiation tRNA-like structure, which mediates the binding of ribosomes to RNA and initiates protein translation. This non-coding RNA sequence is called the internal ribosome entry site (IRES). In genetic engineering, IRES sequences are often used for multi-gene expression. For example, after the target gene, the IRES sequence can be inserted, followed by a selectable marker gene, so that the transcribed mRNA can express two proteins at the same time.

Tuberculosis (TB) is an infectious disease caused by Mycobacterium tuberculosis and is mainly spread through the respiratory tract. In recent years, tuberculosis prevention and treatment are facing new challenges globally with the emergence of multi-drug resistance strains. Earlier secreted antigenic target-6 (ESAT6), a secretory antigen, and culture filtrate protein 10 (CFP10) are two small molecular proteins synthesized and secreted at the early stage after the host is infected by Mycobacterium tuberculosis, and are specific antigens of pathogenic Mycobacterium tuberculosis, and have T cell epitope that can stimulate T cells immunity. These two small molecular proteins does not exist in Bacille Calmette Guerin vaccine (BCG) strains, and thus can serve as a marker to distinguish BCG inoculation from tuberculosis. They are very important in diagnosing tuberculotic infection and tuberculosis. Naturally, ESAT6 and CFP10 can form a protein dimer. Compared with stimulating with ESAT6 or CFP10 alone, the protein dimer can better stimulate T cells to cause an immune response.

Chinese patent No. CN1603415A discloses a method for fusion-expressing ESAT6 in Pichia pastoris. In this method, ESAT-6 and human α-2a interferon genes were fused together and linked with a human enterokinase recognition sequence. Fusion gene was inserted into an expression vector, and then transformed into Pichia pastoris strains, in which ESAT6 fusion protein was expressed in high level in secretion state. Then, high purity ESAT6 fusion protein could be purified by hydrophobic chromatography and ion exchange columns. However, this monomer ESAT6 fusion protein has a weaker ability to stimulate the T cells, compared with the ESAT6 and the CFP10 together. But recombinant CFP10 is an insoluble protein and has a low expression level in prokaryotic expression system and thus is difficult to obtain by similar methods.

Chinese patent No. CN103146715A discloses a method for several ESAT6 gene fused together, expressed and purified, and use thereof in Tuberculosis detection. In this method, fused ESAT6 also could stimulates a subject's peripheral blood T cells to release γ-interferon (IFN-γ). By measuring the change of γ-interferon, it can be diagnosed whether the subject is infected with Mycobacterium tuberculosis or not. The diagnostic kit disclosed by this patent has high specificity and high sensitivity, etc., and meets the requirement of clinical diagnosis. However, fusion ESAT6 has less simulation capability compare with the native ESAT6-CFP10 dimer, either.

Chinese patent No. CN104628862A discloses a human Mycobacterium tuberculosis fusion protein and use thereof. In this method, the gene sequences of ESAT6, CFP10, and TB7.7 (called ECT for short) are synthesized and directly inserted in series into a plasmid pET28b. The obtained fusion protein contains all the three protein fragments, ESAT6, CFP10, and TB7.7, and can stimulate human T cells to release γ-interferon (IFN-γ). However, in the process of establishing the recombinant plasmid, the exogenous fusion ECT gene sequence inserted has a length of about 1000 bp, and the obtained ESAT6/CFP10/TB7.7 recombinant protein is not highly soluble, cannot form a native conformation, and thus has insufficient stimulatory activity on T cells. Also, introduction of heterologous protein may stimulate T cells and cause errors.

Chinese patent NO. CN105218678A discloses a recombinant ESAT6-CFP10 fusion protein of Mycobacterium tuberculosis and a method for preparing the same. In this method, ESAT6 gene is firstly inserted into a plasmid pET-30a to construct a pET-30a-ESAT6 recombinant plasmid. Then the pET-30a-ESAT6 recombinant plasmid and CFP10 gene are used to construct a pET-30a-ESAT6-CFP10 recombinant plasmid. Then the pET-30a-ESAT6-CFP10 recombinant plasmid is transformed into E. coli DH5α competent cells to obtain the ESAT6-CFP10 fusion protein. The ESAT6-CFP10 fusion protein is not highly soluble, requires protein renaturation treatment during purification, and is not highly yielded. The ESAT6-CFP10 linear fusion sequence is too long, and has low expression efficiency and weaker ability to stimulate T cells than that of the ESAT6-CFP10 protein dimer.

Chinese patent No. CN102191209A discloses a VEGF165-Ang-1 double gene co-expression vector and use thereof. In this double gene co-expression vector pAdTrack-CMV-Ang-1-IRES-VEFG165, human genes VEGF165 and Ang-1 are linked via an internal ribosome entry site (IRES) sequence, resulting in an increased expression level of VEGF165 to a certain extent. However, it is still necessary to transfer the VEGF165 and Ang-1 genes to the vector plasmid successively in two steps in the vector construction process, which is cumbersome and inefficient.

Therefore, it is of great significance to the prevention, early diagnosis, and treatment of tuberculosis to find a simple and effective method for preparing a protein dimer to obtain a soluble ESAT-CFP10 protein dimer in high purity, which has a structure similar to the natural Mycobacterium tuberculosis protein ESAT6-CFP10 and a strong ability to stimulate T cell.

Rheumatoid arthritis (RA) is a chronic systemic inflammatory disease whose hallmark feature is a persistent symmetric polyarthritis (synovitis) and has unknown etiology. Rheumatoid arthritis is characterized by symmetric and aggressive joint inflammation of multiple joints including hand and foot facet joints, often accompanied by involvement of extra-articular organs and positive serum rheumatoid factor, and may lead to deformities and loss of function of the joints. It is characterized by persistent synovitis, systemic inflammation, and the presence of autoantibodies. At present, the etiology and pathogenesis of RA are still poorly understood. Timely diagnosis and treatment are of great significance for controlling RA. At present, there are many clinical methods for diagnosing RA. In American College of Rheumatology (ACR) Classification Criteria for Rheumatoid Arthritis 2010, the number of involved joints, disease courses are specified as well as biological factors (such as RF, CCP, CRP and the like) closely related to RA. However, with the currently used clinical combined diagnosis methods (such as CCP, RF, GPI, AKA, etc.), some RA patients (especially early RA patients) still cannot be detected in time. Therefore, searching for RA-related biomarkers has received continuous attention. RA is an autoimmune disease, and many functional proteins undergo abnormal post-translational modifications during its pathogenesis, such as citrullination, carbamylation, and glycosylation. These abnormally modified proteins can boost the immune system in the body to produce autoantibodies. Studies so far have confirmed that the autoantibody with the highest specificity for RA is an autoantibody against an epitope comprising citrulline. Anti-cyclic citrullinated peptide antibody is an autoantibody against a synthetic cyclic citrullinated peptide (CCP) as an antigen, has high sensitivity and specificity in detecting rheumatoid arthritis (RA) and is a highly specific indicator for early diagnosis of RA. The first-generation artificially synthesized polypeptides are detected with a sensitivity of only about 30% before cyclization, and up to about 50% after cyclization, while the second-generation cyclic peptide can be detected with a sensitivity of about 70%. These CCP peptides form a ring relying only on a disulfide bond. If there is a zipper fastener structure that can increase the stability of the ring, more breakthroughs would be brought in scientific research and applications by this more stable cyclic peptide structure.

### English Abbreviations Table

1. Flag-tag: a tag used for detecting protein expression, composed of 8 amino acids;

2. ESAT6: secretory protein of Mycobacterium tuberculosis;

3. CFP10: secretory protein of Mycobacterium tuberculosis;

4. CCP: cyclic citrullinated peptide, used for diagnosing rheumatoid arthritis;

5. RA: rheumatoid arthritis;

6. K: lysine;

7. D: aspartic acid;

8. IFNγ: interferon-γ, a cytokine;

9. IP10: interferon-inducible protein-10, a chemokine;

10. IL-6: interleukin 6;

11. IL-8: interleukin 8;

12. TNFα: tumor necrosis factor α;

13. PHA: phytohaemagglutinin, a lectin which is found in plants, especially in leguminous plants, and has activities of promoting mitosis and interferon secretion of T cells;

14. IRES: internal ribosome entry site;

15. BCG: Bacillus Calmette Guerin vaccine;

16. ECT gene: fused gene of ESAT6, CFP10, and TB7.7;

17. VEGF165: Human vascular endothelial growth factor;

18. Ang1: angiogenin 1;

19. RF: rheumatoid factor;

20. CRP: C-reaction protein, which may be significantly increased when inflammation or tissue injury occurs, and thus can be used in detection;

21. GPI: glucose-6-phosphate isomerase, used for detecting rheumatoid arthritis;

22. AKA: anti-keratin antibodies, used for detecting rheumatoid arthritis.

The abbreviations not shown above are commonly used abbreviations in the field.

### REFERENCES

Bell S. L., Gongqiao XU, Forstner J. F.. Role of the cystine-knot motif at the C-terminus of rat mucin protein Muc2 in dimer formation and secretion [J]. Biochemical Journal, 2001, 357(1): 203-9.

Elliott J. M, Ultsch M Lee J., et al. Antiparallel conformation of knob and hole aglycosylated half-antibody homodimers is mediated by a CH₂-CH₃ hydrophobic interaction [J]. Journal of molecular biology, 2014, 426(9): 1947-1957.

Sherilyn L. h, 2001h, Role of the cystine-knot motif at the C-terminus of rat mucin protein Muc2 in dimer formation and secretion. Biochemical Journal Jul. 1, 2001, 357(1): 203-209.

### SUMMARY

An object of the present disclosure is to overcome at least one defect in conventional art and to provide a method for promoting formation of protein dimer or cyclic peptide.

In a first aspect of the present disclosure, the present disclosure provides a method for promoting formation of a protein dimer or a cyclic peptide, including introducing a dimer zipper fastener part into the terminal part of a peptide chain, wherein the dimer zipper fastener part is characterized in that:

1) the dimer zipper fastener part includes at least 2 charged amino acid residues, preferably 2 to 9, 3 to 8, 3 to 7, or 4 to 6 charged amino acid residues;
2) the dimer zipper fastener part includes uncharged spacers, wherein the spacer is 1 to 5 amino acids in length, preferably 1 to 4, 1 to 3, or 1 to 2 amino acids in length;
3) at least one of the spacers includes at least one cysteine residue, preferably 1 to 4, 1 to 3, or 1 to 2 cysteine residues; and
4) two dimer zipper fastener parts are bound together by electrostatic interaction of charged amino acids and disulfide bonds are formed via the cysteine residues in the spacers.

For the protein dimer, at least one dimer zipper fastener part is introduced into each peptide chain of the two peptide chains of the protein dimer. The dimer zipper fastener part can be directly conjugated to the peptide chain, or indirectly via a linker peptide. On the premise of not affecting activities of the protein dimer and facilitating the proximity of the two dimmer zipper fastener parts to each other, an appropriate approach for introducing the dimer zipper fastener parts can be selected according to actual needs. Preferably, the dimer zipper fastener part is introduced by indirect conjugation via a linker peptide.

For the cyclic peptide, the dimer zipper fastener parts are introduced into two ends of a peptide chain to be cyclized. The approach and principle for introducing the dimer zipper fastener part into the peptide chain to be cyclized are the same as those for the protein dimer.

The disulfide bond can be formed via cysteine residues between two dimer zipper fastener parts, and can further improve the stability of the dimer zipper fastener.

The spacer can be provided to prevent the charged amino acids from unduly concentrated, thereby preventing local polarity from changing too much and reducing the influence of the charged amino acids on the original peptide chain. The spacer can further facilitate bending of dimer zipper fastener part and binding between zipper fastener parts. The amino acids in the spacer can be foldable small amino acids such as glycine, serine, alanine, and the like. When the amino acids in the spacer are hydrophobic amino acids such as leucine, isoleucine, valine, and the like, binding of the dimer zipper fastener parts can be enhanced by hydrophobic interaction. A number of the spacer can be 1, 2, 3, or more.

A disulfide bond (a dimer fastener) formed by the interaction between cysteines can increase the stability of the dimer or the cyclic peptide. The more disulfide bond there is, the higher stability of the corresponding dimer zipper fastener has. The number of the disulfide bond can be increased according to actual needs without affecting the activity of the protein or peptide. However, too many cysteine residues in one spacer can affect pairing, thus the number of the cysteine residues in one spacer is preferably no more than two pairs.

On the premise of facilitating the formation of protein dimer or cyclic peptide, the length of the dimer zipper fastener part can be determined according to actual needs. The length of the dimer zipper fastener part can be 3 aa at the minimum and can range from 3 aa to 20 aa.

Examples of some protein dimers obtained in the first aspect of the present disclosure are shown in FIG. 1. The dimer zipper fastener may have the following features:

(1) In some dimer zipper fastener parts, charged amino acids are symmetrically located at both sides of a spacer (symmetry point), and the spacer is a cysteine (C) residue; two dimer zipper fastener parts are bound by electrostatic interaction of positive and negative charges; a disulfide bond is formed by the reaction of —SH groups of the cysteine residues, serving as a “fastener” that combines two protein chains or peptide chains to form a protein dimer (in FIG. 1-1, FIG. 1-3 and FIG. 1-6).

(2) In some dimer zipper fastener parts, charged amino acids are asymmetrically located at both sides of a spacer, and the spacer is a cysteine (C) residue; two dimer zipper fastener parts are bound by electrostatic interaction of positive and negative charges; a disulfide bond is formed by the reaction of —SH groups of cysteine residues, serving as a “fastener” that combines two protein chains or peptide chains to form a protein dimer (in FIG. 1-2, FIG. 1-4 and FIG. 1-5).

(3) Charged amino acids are symmetrically located at both sides of a spacer containing cysteines, and a number of the charged amino acids is 2 to 9 (FIG. 1-1).

(4) Charged amino acids are asymmetrically located at both sides of a spacer containing cysteines, and the dimer zipper fastener parts are all located at N-terminus of the target protein (FIG. 1-2).

(5) Charged amino acids are symmetrically located, and the dimer zipper fastener parts are all located at C-terminus of the target protein (FIG. 1-3).

(6) Charged amino acids are asymmetrically located, and the dimer zipper fastener parts are all located at C-terminus of the target protein (FIG. 1-4).

(7) Charged amino acids are crosswise located, and the dimer zipper fastener parts are all located at C-terminus of the target protein (FIG. 1-5).

(8) A dimer of a native protein has an end-to-end structure, and a dimer zipper fastener part including a linker peptide can be located at N-terminus and C-terminus of the target protein, respectively, thus a right three-dimensional structure of proteins can also be formed (FIG. 1-6).

FIG. 2 is a structural schematic diagram of a cyclic peptide with a dimer zipper fastener part. In this dimer zipper fastener parts, charged amino acids are symmetrically located at both sides of a spacer (symmetry point), and the spacer is a cysteine (C) residue; two dimer zipper fastener parts are bound by electrostatic interaction of positive and negative charges; a disulfide bond is formed by the reaction of —SH groups of the cysteine residues, serving as a “fastener” that combines two ends of the peptide chain to form a cyclic peptide. The dimer zipper fastener parts for forming the cyclic peptide can be symmetrically or asymmetrically located, or can be crosswise located.

In some embodiments, the charged amino acids are symmetrically or asymmetrically located at both sides of the spacer. The charged amino acids on both sides of the spacer can regulate the direction and strength of the formed dimer zipper fastener by various permutations and combinations. When the charged amino acids are symmetrically located at both sides of the spacer, the two complementary dimer zipper fastener parts can be combined in two different directions, which facilitate forming a protein dimer or a cyclic peptide. However, it would cause inaccurate regulation of the conformation of protein dimers, that is, the peptide chain can be located at the same side or different side of the dimer zipper fastener in the obtained protein dimer. When the charged amino acids are asymmetrically located at both sides of the spacer, the two complementary dimer zipper fastener parts can be combined in a certain direction, which facilitates controlling the conformation of the protein dimer, so as to ensure that the two peptide chains have an expected conformation. In theory, the electrostatic affinity between the two complementary dimer zipper fastener parts enhances as the number of complementary paired charged amino acids therebetween increases.

Structures of the protein dimers in some embodiments are shown in FIG. 3. In some dimer zipper fastener parts, charged amino acids are asymmetrically located at both sides of a spacer, and the spacer is a cysteine (C) residue. Two dimer zipper fasteners are bound by electrostatic interaction of positive and negative charges in a certain direction. A disulfide bond is formed by the reaction of —SH groups of cysteine residues, serving as a “fastener” that combines two protein chains or peptide chains to form a protein dimer. When each of two proteins or two polypeptides has a dimer zipper fastener part, the two proteins or two polypeptides can form the protein dimer or an extended peptide.

In some embodiments, the charged amino acids are positive-charged amino acids or negative-charged amino acids. The positive-charged amino acid is selected from the group consisting of lysine (K), arginine (R), and histidine (H). The negative-charged amino acid is selected from the group consisting of aspartic acid (D) and glutamic acid (E).

In some embodiments, N-terminus and C-terminus of at least one of the peptide chains are linked to the dimer zipper fastener part; specifically, the N-terminus and the C-terminus of one peptide chains are linked to the dimer zipper fastener part, respectively. Respectively introducing the dimer zipper fastener parts to both ends of the peptide chain can further extend the length of the peptide chain, thus obtaining a longer peptide chain.

In some embodiments, at least one of the peptide chains includes a tag sequence; preferably, each of two peptide chains includes one tag sequence; and, preferably, the tag sequence includes a Flag-tag sequence and a histidine sequence. Introducing the tag sequence can facilitate the separation and purification of the target protein.

In some embodiments, one peptide chain of the protein dimer is ESAT6, the other peptide chain is CFP10.

Preferably, the dimer zipper fastener is located at C-terminus of ESAT6 and CFP10, respectively.

The cyclic peptide includes a CCP linear amino acid sequence, and the dimer zipper fastener parts are located at N-terminus and C terminus of the CCP linear amino acid sequence, respectively.

Preferably, the dimer zipper fastener part includes 4 charged amino acid residues, wherein the positive-charged amino acid is preferably lysine or aspartic acid, and the dimer zipper fastener includes 1 or 2 cysteine residues.

Preferably, an integral structure of the cyclic peptide is KKCK-CCP linear amino acid sequence-DCDD.

In a second aspect of the present disclosure, the present disclosure provides a protein or polypeptide with a dimer zipper fastener, wherein at least one of the terminal part of the protein or polypeptide is linked to the dimer zipper fastener part, wherein the dimer zipper fastener part is characterized in that: In a first aspect of the present disclosure, the present disclosure provides a method for promoting formation of a protein dimer or a cyclic peptide, including introducing a dimer zipper fastener part into the teiminal part of a peptide chain, wherein the dimer zipper fastener part is characterized in that:

1) the dimer zipper fastener part includes at least 2 charged amino acid residues, preferably 2 to 9, 3 to 8, 3 to 7, or 4 to 6 charged amino acid residue;
2) the dimer zipper fastener part includes uncharged spacers, wherein the spacer is 1 to 5 amino acids in length, preferably 1 to 4, 1 to 3, or 1 to 2 amino acids in length;
3) at least one of the spacers includes at least one cysteine residue, preferably 1 to 4, 1 to 3, or 1 to 2 cysteine residues; and
4) two dimer zipper fastener parts are bound by electrostatic interaction of charged amino acids and disulfide bonds are formed via the cysteine residues in the spacers.

In some embodiments, the charged amino acids are symmetrically or asymmetrically located at both sides of the spacer.

In some embodiments, the protein is ESAT6 and CFP10; and the dimer zipper fastener parts are located at C-terminus of ESAT6 and C-terminus of CFP10.

The protein is CCP linear amino acid, and the dimer zipper fastener parts are located at N-terminus and C-terminus of the CCP linear amino acid sequence, respectively.

Preferably, the dimer zipper fastener part includes charged amino acid residues with 4 charges, wherein the charged amino acid is preferably selected from the group consisting of lysine and aspartic acid, respectively, and the dimer zipper fastener part includes 1 or 2 cysteine residues.

Preferably, an integral structure of the cyclic peptide is KKCK-CCP linear amino acid sequence-DCDD.

In a third aspect of the present disclosure, the present disclosure provides an expression vector including a nucleotide sequence, wherein the nucleotide sequence is able to express the peptide chain including the dimer zipper fastener of the first aspect and the second aspect of the present disclosure.

The expression vector can be any of the well-known vectors without limitation.

In a fourth aspect of the present disclosure, the present disclosure provides a method for preparing a zipper fastener-type protein dimer or a cyclic peptide, including:

1) constructing an expression vector, wherein the expression vector is as described in the third aspect of the present disclosure; and,
2) expressing by transforming into an expression strain or cell with the expression vector, then isolating, and purifying the zipper fastener-type protein dimer or the cyclic peptide.

In some embodiments, the peptides in the zipper fastener-type protein dimers are ESAT6 and CFP10, respectively.

In some embodiments, expression of ESAT6 gene and CFP10 gene include their own initiation codons and termination codons; the genes are inserted into two expression vectors, respectively, and are simultaneously transfected into strains or cells for expression.

In some embodiment, expression of ESAT6 gene and CFP10 gene include their own independent initiation codons and termination codons; the genes are inserted into the same expression vector and are separated by a spacer sequence.

In some embodiments, the spacer sequence is an IRES sequence.

In some embodiments, the modified ESAT6 has a sequence as follows:

(SEQ ID NO.: 1)
MAEMKTDAATLAQEAGNFERISGDLKTQIDQVESTAGSLQGQWRGAAG
TAAQAAVVRFQEAANKQKQELDEISTNIRQAGVQYSRADEEQQQALSS
QMGFGGDDCDD.

In some embodiments, the modified CFP10 has a sequence as follows:

(SEQ ID NO.: 2)
MTEQQWNFAGIEAAASAIQGNVTSIHSLLDEGKQSLTKLAAAWGGSGS
EAYQGVQQKWDATATELNNALQNLARTISEAGQAMASTEGNVTGMFAG
GKKCKK.

In some embodiments, at least one of ESAT6 and CFP10 contains a tag sequence.

In some embodiments, each of the two proteins contains the tag sequence.

In some embodiments, the tag sequence contains a Flag-tag sequence and a His-tag sequence. The Flag-tag sequence is DYKDDDDKGG (SEQ ID NO.: 3), and the His-tag sequence is HHHHHHGG (SEQ ID NO.: 4).

In a fifth aspect of the present disclosure, the present disclosure provides use of the zipper fastener-type ESAT6-CFP10 protein dimer in preparing a detection reagent, wherein the detection reagent is configured to detect M. tuberculosis-specific T cell immune response in a sample, to diagnose whether a subject is infected with M. tuberculosis, to evaluate therapeutic effect of antituberculous or to analyze infective mechanism of M. tuberculosis; and, the zipper fastener-type ESAT6-CFP10 protein dimer has the structure of the ESAT6-CFP10 protein dimer disclosed in the first aspect and the second aspect of the present disclosure.

In a sixth aspect of the present disclosure, the present disclosure provides a kit, including the zipper fastener-type ESAT6-CFP10 protein dimer disclosed in the first aspect or the second aspect of the present disclosure.

In some embodiments, the kit further includes a reagent for measuring a level of a cytokine, wherein the cytokine is at least one selected from the group consisting of IFNγ, IP10, IL-6, IL-8, and TNFα.

In some embodiments, the kit further includes a blood sampling device.

In some embodiments, the kit further includes a positive control tube containing endotoxin free PHA.

In some embodiments, the kit further includes a negative control tube containing control reagents without the zipper fastener-type EAST6-CFP10 protein dimer.

In a seventh aspect of the present disclosure, the present disclosure provides use of the zipper fastener-type CCP cyclic peptide in preparing a detection reagent, wherein the detection reagent is configured to detect an anti-citrullinated protein autoantibody in serum of a rheumatoid arthritis patient; wherein the zipper fastener-type CCP cyclic peptide has a structure of CCP cyclic peptide disclosed in the first aspect and the second aspect of the present disclosure.

The advantages of the present disclosure are shown hereinafter.

In the dimer zipper fastener parts in some embodiments of the present disclosure, due to the charged amino acid groups, the dimer zipper fastener parts carrying identical charges would repel, and only carrying opposite charges can bind with each other. This further locates the position of the cysteine, and can reduce the possibility of forming a multimer via the disulfide bonds of the cysteine.

In some embodiments of the present disclosure, the dimer zipper fastener parts can effectively facilitate forming of dimers between proteins, stabilize the formed protein dimer, and allow the proteins naturally having dimerization tendency to form a stable structure.

Also, the protein dimers in some embodiments of the present disclosure can be obtained by the conventional recombinant protein expression method. The dimer can be formed using an expression system. The same tag for isolation can be used to isolate the obtained polypeptide or protein polymer, greatly improving the efficiency of isolation and purification of the protein.

In some embodiments of the present disclosure, since the dimer zipper fastener parts help proteins to form a stable structure, the expression level of the integral protein can be improved and solubilities of the two proteins can also be improved, which simplifies the purification of protein dimer and improves the purity of the purified protein.

In some embodiments of the present disclosure, a dimer having a conformation similar to native ESAT6-CFP10 dimer can be obtained. The dimer has better solubility. Compared with the ESAT6-CFP10 linear fusion protein, the dimer zipper fastener has a better stimulation effect to memory T cells.

In some embodiments of the present disclosure, the dimer zipper fasteners help the synthesized polypeptide to form a stable cyclic structure. For example, the stable cyclic structure can facilitate recognition of rheumatoid arthritis-specific autoantibodies by CCP peptides, and increase the sensitivity of detection. This polypeptide can be used in a kit for detecting rheumatoid arthritis.

In some embodiments of the present disclosure, the dimer zipper fastener parts are added at both ends of a conventional CCP amino acid sequence to replace the original disulfide bond, so as to obtain a zipper fastener-type CCP cyclic peptide. The zipper fastener-type cyclic peptide can enhance the stability of the formed cyclic peptide. The zipper fastener-type cyclic peptide can be immobilized on solid support as an antigen for detecting anti-citrullinated protein autoantibody in serum of a rheumatoid arthritis patient. The solid support is any one or a combination of at least two of an ELISA plate, magnetic beads, an affinity membrane, or a liquid-phase chip. The kit further includes an enzyme-labeled human antibody, a negative control substance, a positive control substance, a critical control substance, a sample diluent, a blocking buffer, a washing buffer, a substrate solution and a stop buffer.

### BRIEF DESCRIPTION OF THE DRAWINGS

FIG. 1 is a schematic diagram of protein dimers with dimer zipper fasteners, wherein: 1) charged amino acids are symmetrically located at both sides of the spacer containing cysteines, and a number of the charged amino acids is 2 to 9; 2) charged amino acids are asymmetrically located at both sides of the spacer containing cysteines, and are all located at N-terminus of a target protein; 3) charged amino acids are symmetrically located, and the dimer zipper fastener parts are located at C-terminus of a target protein; 4) charged amino acids are asymmetrically located, and the dimer zipper fastener parts are located at C-terminus of a target protein; 5) charged amino acids are crosswise located, and the dimer zipper fastener parts are located at C-terminus of a target protein; and 6) a dimer of a native protein has an end-to-end structure, and a dimer zipper fastener part including a linker peptide can be located at N-terminus and C-terminus of the protein, respectively, thus a right three-dimensional structure conformation can also be formed.

FIG. 2 is a schematic diagram of cyclic peptides with dimer zipper fastener parts. The dimer zipper fastener parts for forming the cyclic peptide can be symmetrically located or asymmetrically located, or can be crosswise located.

FIG. 3 is a schematic diagram of a peptide chain dimer with a dimer zipper fastener and an extended peptide including a dimer zipper fastener.

FIG. 4 is an SDS-PAGE pattern of zipper fastener-type protein dimers expressed by different IPTG-induced strains.

FIG. 5 is a Coomassie brilliant blue-stained SDS-PAGE gel pattern of a purified zipper fastener-type protein dimer. SDS and reducing agent B-mecaptoethanol broke the disulfide bond, resulting in the decomposition of a native protein dimer into two monomers, while the protein before the reduction was clearly in a dimeric state.

FIG. 6 shows the effect of zipper fastener-type protein dimer antigens at various concentrations on IFN-γ stimulation levels. The zipper fastener-type protein dimer had relatively strong stimulation activity, and can reach saturation at a relatively lower concentration.

FIG. 7 shows the effect of stimulating temperature on T cell secreting IFN-γ.

FIG. 8 shows the effect of stimulating time on T cell secreting IFN-γ.

FIG. 9 shows the effect of proteins having different structures on T cell secreting IFN-γ, a zipper fastener-type protein dimer has a stronger activity. The zipper fastener-type protein dimer had a stronger effect on stimulating T cells to secrete IFN-γ at the same concentration as the protein dimer without a zipper fastener structure, and thus the T cells secreted more IFN-γ.

FIG. 10 shows the comparison of sensitivity and specificity of zipper fastener-type CCP cyclic peptide with a conventional disulfide bond CCP cyclic peptide in diagnosing rheumatoid arthritis.

FIG. 11 shows the performance of the zipper fastener-type CCP cyclic peptide and the conventional CCP cyclic peptide in testing 26 different rheumatoid arthritis samples. S/CO values were higher in 12 samples tested by zipper fastener-type CCP cyclic peptide, which turned a negative result (undetectable) to a positive result (detectable).

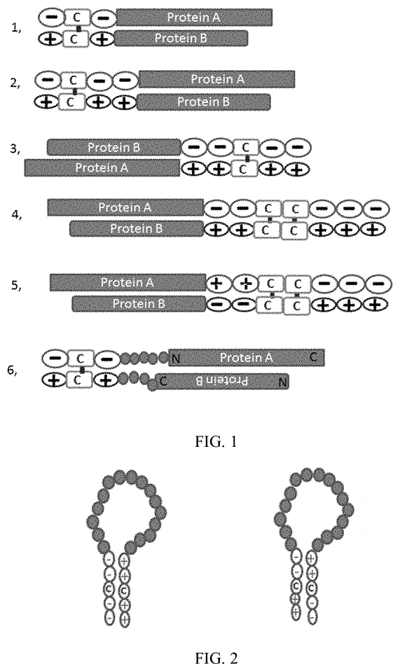

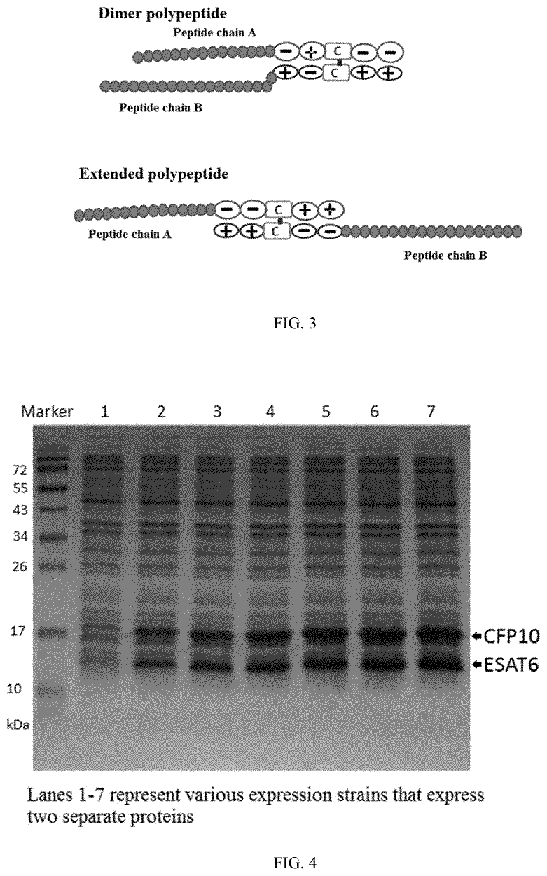

### DETAILED DESCRIPTION

The technical solutions of the present disclosure will be further explained below in conjunction with the examples. The specific examples described herein are only for the purpose of interpreting the present disclosure, but are not to limit the present disclosure. In addition, it should be noted that, for ease of description, the drawings only show a part of the structure related to the present disclosure, instead of all the structures.

The specific techniques or conditions not specified in the embodiments are carried out in accordance with the techniques or conditions described in the literature in the field, or in accordance with the product instructions. All the reagents or instruments not indicated with the specific manufacturer are conventional products that are commercially available.

### EXAMPLE 1

### Construction of Zipper Fastener-Type ESAT6-CFP10 Protein Dimer Expression Vector

An amino acid set with negative charges was added to C-terminus of ESAT6 gene, and an amino acid set with positive charges was added to C-terminus of CFP10 gene. A His-tag for purification was added at the front of the CFP10 gene.

An expressed amino acid sequence of the ESAT6 was DYKDDDDKGG-MAEMKTDAATLAQEAGNFERISGDLKTQIDQVESTAGSLQGQWRGAAGTAAQAAV VRFQEAANKQKQELDEISTNIRQAGVQYSRADEEQQQALSSQMGF-GGDDKDD.

An expressed amino acid sequence of the CFP10 was: HHHHHHGG-MTEQQWNFAGIEAAASAIQGNVTSIHSLLDEGKQSLTKLAAAWGGSGSEAYQGVQQ KWDATATELNNALQNLARTISEAGQAMASTEGNVTGMFA-GGKKCKK.

ESAT6 and CFP10, to which the above-mentioned sequences had been added, were inserted to a pET expression vector at both ends of IRES sequence, respectively, thus obtaining an expression plasmid. After purification, the expression plasmid was transformed into BL21(DE3) competent cells.

### Expression and Purification of the Recombinant Protein

Successfully constructed single colonies were selected and cultured at 37° C. in 2 liters culture. After IPTG induction for 4 h, the cells were centrifuged, collected, and disrupted by ultrasonic. After Ni-column affinity purification, obtain the interest protein, i.e. zipper fastener-type protein dimer ESAT6-CFP10 (named E6C10 for short).

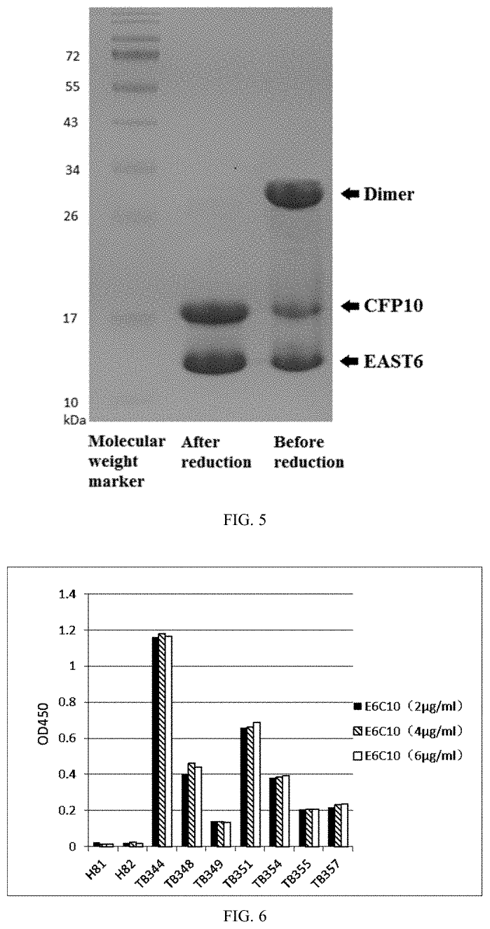

The protein was measured for concentration by SDS-PAGE. The result is shown in FIG. 4. It can be seen from FIG. 4 that a soluble dimer could be obtained 4 hours after induction at 37° C. It can be seen from FIG. 5 that the soluble dimer could be further purified by Ni affinity column to achieve a purity above 90%. The purified protein was subjected to endotoxin-removing treatment before a memory T cell stimulation test.

### Memory T Cell Stimulation Test for the Zipper Fastener-Type Protein Dimer

In this experiment, fresh peripheral whole blood from tuberculosis patients was used to compare the effect of the zipper fastener dimer antigens in different concentrations (2 μg/ml, 4 μg/ml and 6 μg/ml) on stimulation levels (i.e., IFN-γ levels).

1) Nine fresh peripheral whole blood samples were collected from two healthy people and seven tuberculosis patients, and each of the whole blood samples was divided into 5 aliquots (1 mL for each). Stimulation was performed using the zipper fastener dimer protein in three different concentrations, a negative control substance, and a positive control substance.

2) 45 samples were incubated dormant at 37° C. for 20 hours.

3) Samples were centrifuged to collect plasma supernatants.

4) IFN-γ levels were measured by Human IFN-γ Elisa kit (a standard double antibody sandwich ELISA kit with lowest detectable concentration of IFN-γ at 5 pg/mL).

The test results were shown in Table 1 and FIG. 6 (the results of the negative control and the positive control were not shown).

TABLE 1
Effect of the zipper fastener-type protein dimer antigen in
different concentrations on IFN-γ stimulation level.

| | Protein | Protein | Protein |
|---|---|---|---|
| | dimer | dimer | dimer |
| Sample No. | (2 μg/ml) | (4 μg/ml) | (6 μg/ml) |
| Healthy person 81 | 0.023 | 0.017 | 0.017 |
| Healthy person 82 | 0.021 | 0.026 | 0.019 |
| TB344tubercu1osis | 1.162 | 1.182 | 1.168 |
| TB348tuberculosis | 0.405 | 0.463 | 0.442 |
| TB349tuberculosis | 0.143 | 0.14 | 0.135 |
| TB351tuberculosis | 0.657 | 0.665 | 0.688 |
| TB354 tuberculosis | 0.38 | 0.386 | 0.395 |
| TB355tuberculosis | 0.206 | 0.209 | 0.207 |
| TB357tuberculosis | 0.219 | 0.233 | 0.237 |

It can be seen from FIG. 6 that, in the test to the seven tuberculosis patients and two healthy people, IFN-γ level of the samples from the tuberculosis patients were approximately plateaued upon stimulating the cells with the zipper fastener type protein dimer antigen obtained in Example 1 in 2 μg/mL, and cannot be further increased (except for two samples in which IFN-γ level was slightly increased) even if concentrations of the zipper fastener type protein dimers were increased to 4 μg/mL and 6 μg/mL. In the following tests of the present disclosure, stimulation was performed using the antigens in a concentration of 2 μg/ml.

### Selecting Culturing Temperature for Zipper Fastener-Type Protein Dimer Stimulated Cells

In this experiment, effects of culturing temperatures (25° C., 30° C., 37° C., 38° C., and 39° C.) were tested in fresh peripheral whole blood of tuberculosis patients according to a detailed process as follows:

1) Fresh peripheral whole blood samples were collected from four tuberculosis patients, and each of samples was divided into 10 aliquots. For 5 aliquots, stimulation was performed using the zipper fastener dimer (in a final concentration of 2 μg/mL). As for the other 5 aliquots, a negative control substance was used.

2) The samples from the 4 patients were incubated at 25° C., 30° C., 37° C., 38° C., and 39° C. for 22 hours.

3) The samples as well as the negative control were centrifuged to collect plasma supernatants.

4) The IFN-γ level in the plasma supernatants were measured by Human IFN-γ Elisa kit (a standard double antibody sandwich ELISA kit with a lowest detectable concentration of IFN-γ at 5 pg/mL).

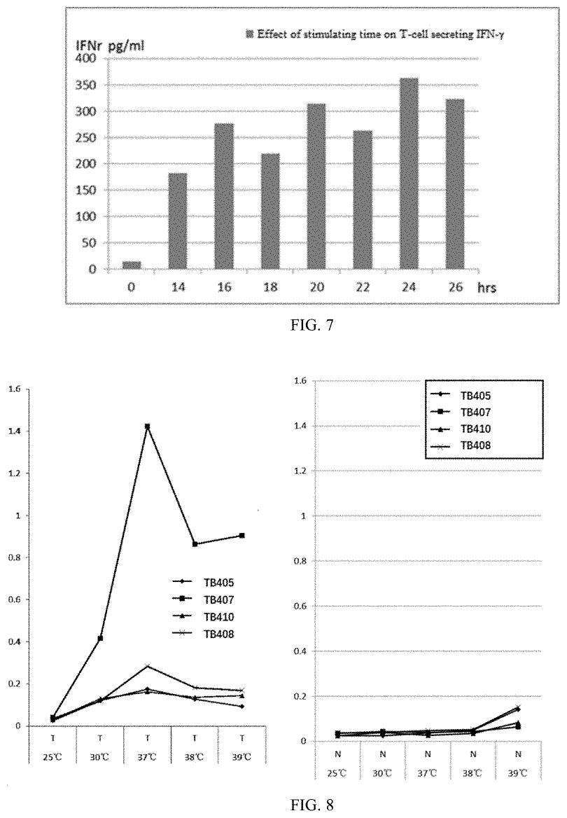

The test results were shown in Table 2 and FIG. 7.

TABLE 2
Effect of culturing temperature on IFN-γ expression

| | 25° C. | | 30° C. | | 37° C. | | 38° C. | | 39° C. | |
|---|---|---|---|---|---|---|---|---|---|---|
| | Negative | | Negative | | Negative | | Negative | | Negative | |
| Tuberculosis | 0.052 | 0.026 | 0.025 | 0.121 | 0.038 | 0.126 | 0.050 | 0.127 | 0.142 | 0.092 |
| TB405 | | | | | | | | | | |
| Tuberculosis | 0.044 | 0.041 | 0.136 | 0.417 | 0.069 | 1.423 | 0.053 | 0.864 | 0.04 | 0.904 |
| TB407 | | | | | | | | | | |
| Tuberculosis | 0.024 | 0.029 | 0.039 | 0.03 | 0.017 | 0.032 | 0.026 | 0.036 | 0.024 | 0.044 |
| TB410 | | | | | | | | | | |
| Tuberculosis | 0.038 | 0.034 | 0.039 | 0.118 | 0.047 | 0.284 | 0.057 | 0.182 | 0.152 | 0.168 |
| TB408 | | | | | | | | | | |

It can be seen from FIG. 7 that the IFN-γ level upon stimulation varies at the culturing temperature between 30° C. and 38° C. The INF-γ level upon stimulation was the highest at the culturing temperature of 37° C., revealing the best stimulation effect. No stimulation effect was shown at the culturing temperature of 25° C. Nonspecific response was observed in some of the negative control samples at the culturing temperature of 39° C. Therefore, in the present disclosure, the culturing temperature may range from 30° C. to 38° C., preferably 37° C.

### Selecting Culturing Time for Zipper Fastener Type Protein Dimer Stimulated Cells

In this experiment, effects of culturing times (14 h, 18 h, 20 h, 22 h, 24 h, 26 h) on stimulation levels (i.e., IFN-γ stimulation levels) were assessed in fresh peripheral whole blood of tuberculosis patients.

1) Fresh peripheral whole blood was collected from tuberculosis patients. Stimulation was performed using the zipper fastener protein dimer (in a final concentration of 2 μg/mL) in the whole blood sample.

2) The whole blood sample upon stimulation was incubated at 37° C. for 12 h, 16 h, 18 h, 20 h, 22 h, 24 h, 26 h, and 28 h. The whole blood sample without stimulation was incubated under the same conditions and serves as a negative control.

3) The samples as well as the negative control were centrifuged to collect plasma supernatants.

4) The samples were measured for IFN-γ level using Human IFN-γ Elisa kit (a standard double antibody sandwich ELISA kit with the lowest detectable concentration of IFN-γ at 5 pg/mL).

The test results are shown in FIG. 8. It could be seen from FIG. 8 that the stimulation level (i.e., IFN-γ level) approximately plateaued after incubation for 20 h. Therefore, in the present disclosure, a preferably culturing time was 20 h. The experiment result was not affected when the culturing time was 20 h plus or minus 2 to 4 h.

### Effect of Zipper Fastener Type Protein Dimer and Linear Fusion Protein on Stimulation Level

In this experiment, fresh peripheral whole blood from tuberculosis patients was used to compare the effect of the zipper fastener type protein dimer and linear fusion protein on stimulation level (i.e., IFN-γ stimulation levels).

1) Fresh peripheral blood samples were collected from seven tuberculosis patients, and each of the whole blood samples was stimulated with the zipper fastener type protein dimer and a linear fusion protein (in a final concentration of 2 μg/mL), respectively.

2) The stimulated whole blood samples were incubated at 37° C. for 20 h. The whole blood samples upon no stimulation by a stimulus were incubated under the same conditions and serves as a negative control.

3) The samples as well as the negative control were centrifuged to collect plasma supernatants.

4) The samples were measured for IFN-γ level using Human IFN-γ Elisa kit (a standard double antibody sandwich ELISA kit with the lowest detectable concentration of IFN-γ at 5 pg/mL).

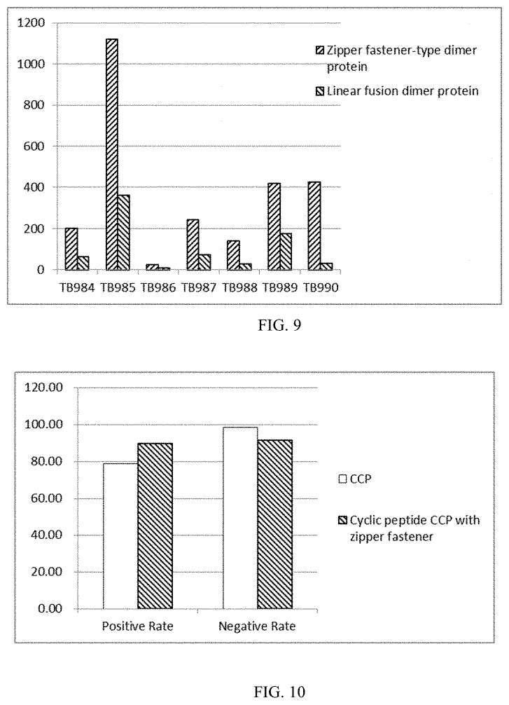

The test results are shown in FIG. 9. It could be seen from FIG. 9 that compared with the linear fusion protein antigen, the zipper fastener-type protein dimer antigen had a significantly better stimulation effect, and more IFN-γ was produced in the whole blood sample stimulated with the zipper fastener-type protein dimer.

In conclusion, in the present disclosure, it can be concluded by investigating stimulation conditions that there is the highest cytokine level, the best stimulation effect and thus the best detection effect, when a concentration of the zipper fastener-type protein dimer was 2 μg/mL and cells are stimulated at a stimulating temperature of 37° C. for 20 h to 22 h.

### EXAMPLE 2: USE OF CYCLIC PEPTIDE IN CCP DETECTION

Based on conventional CCP, positions of the disulfide bond were changed, and dimer zipper fasteners on both sides of the polypeptide were added, then a new structure of the polypeptide was generated as follows: KKCK-CCP-DCDD. With the zipper fastener cyclic peptide, the positive rate of detecting autoimmune antibody in rheumatoid arthritis serum was increased by 10%, indicating that stability of cyclization is very important to detection sensitivity of CCP, and improvement of stability of the cyclic peptide can further increase the detection sensitivity of CCP in detecting an anti-citrullinated protein autoantibody.

A plate was coated with a streptavidin-dimer zipper fastener cyclic peptide CCP (in a concentration of 5 μg/ml), and blocked with skimmed milk. HRP-goat anti-human antibody as a secondary antibody was used for testing a sample. CCP ELISA assay kit (Euro Diagnostica) was used as a control.

Referring to FIG. 10, in 95 RA patients, the positive rate for CCP without the dimer zipper fastener was 78%, and a positive rate for CCP with the dimer zipper fastener was 89%. In 71 healthy people, specificity of CCP with the dimer zipper fastener was 91%, which is slightly lower than that of CCP (98%).

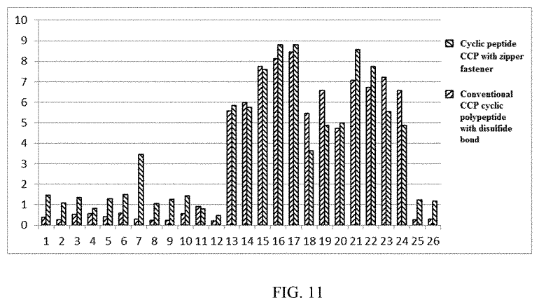

FIG. 11 shows the performance of the zipper fastener-type CCP cyclic peptide and the conventional CCP cyclic peptide in testing 26 different rheumatoid arthritis samples. S/CO values were higher in 12 samples tested by zipper fastener type CCP cyclic peptide, which turns a negative result (undetectable) to a positive result (detectable).

The above description is only the preferred embodiments of the present disclosure and the applied technical principles. One of ordinary skill in the art will understand that the present disclosure is not limited to the specific embodiments described herein. For one of ordinary skill in the art, various obvious changes, adjustments, and substitutions can be made without departing from the protection scope of the present disclosure. Therefore, although the present disclosure has been described in more detail through the above embodiments, the present disclosure is not limited to the above embodiments. Without departing from the concept of the present disclosure, more other equivalent embodiments may be included. The scope of the present disclosure is defined by the scope of the appended claims.

1. A method for promoting formation of a protein dimer or a cyclic peptide, comprising introducing a dimer zipper fastener part into the terminal part of a peptide chain, wherein:
1) the dimer zipper fastener part comprises at least 2 charged amino acid residues;
2) the dimer zipper fastener part comprises uncharged spacers, wherein the spacer is 1 to 5 amino acids in length;
3) at least one of the spacers comprises at least one cysteine residue; and
4) two dimer zipper fastener parts are bound by electrostatic interaction of charged amino acids and disulfide bonds are formed via the cysteine residues in the spacers.

2. The method according to claim 1, wherein the charged amino acids are symmetrically or asymmetrically located at both sides of the spacer.

3. The method according to claim 2, wherein the charged amino acids are positive-charged amino acids or negative-charged amino acids, wherein the positive-charged amino acids are selected from the group consisting of lysine, arginine, and histidine, and the negative-charged amino acids are selected from the group consisting of aspartic acid and glutamic acid.

4. The method according to claim 1, wherein N-terminus and C-terminus of at least one of the peptide chains are linked to the dimer zipper fastener part, respectively.

5. The method according to claim 1, wherein at least one of the peptide chains comprises a tag sequence; preferably, each of two peptide chains of comprises one tag sequence.

6. The method according to claim 1, wherein
one peptide chain of the protein dimer is ESAT6, the other peptide chain is CFP10;
the cyclic peptide comprises a CCP linear amino acid sequence, and the dimer zipper fastener parts are located at N-terminus and C terminus of the CCP linear amino acid sequence, respectively.

7. A protein or polypeptide comprising a dimer zipper fastener, wherein
at least one of the terminal parts of the protein or polypeptide is linked to the dimer zipper fastener part, wherein the dimer zipper fastener part is characterized in that:
1) the dimer zip per fastener part comprises at least 2 charged amino acid residues;
2) the dimer zipper fastener part comprises uncharged spacers, wherein the spacer is 1 to 5 amino acids in length;
3) at least one of the spacers comprises at least one cysteine residue, preferably; and
4) two dimer zipper fastener parts are bound by electrostatic interaction of charged amino acids and disulfide bonds are formed via the cysteine residues in the spacers.

8. The protein or polypeptide according to claim 7, wherein the charged amino acids are symmetrically or asymmetrically located at both sides of the spacer.

9. The protein or polypeptide according to claim 7, wherein
the protein is constructed of ESAT6 and CFP10;
the dimer zipper fastener parts are located at C-terminus of ESAT6 and C-terminus of CFP10.

10. An expression vector, comprising a nucleotide sequence,
wherein the nucleotide sequence is able to express the peptide chain comprising the dimer zipper fastener part according to claim 8.

11. A method for preparing a zipper fastener-type protein dimer or a cyclic peptide, comprising,
constructing an expression vector, wherein the expression vector is the expression vector according to claim 10; and,
expressing by transforming into an expression strain or cell with the expression vector, then isolating, and purifying the zipper fastener-type protein dimer or the cyclic peptide.

12. The method of claim 11, wherein
the peptides in the zipper fastener-type protein dimers are ESAT6 and CFP10, respectively.

13. The method of claim 12, wherein
at least one of the ESAT6 and the CFP10 comprises a tag sequence.

14. (canceled)

15. A kit, wherein the kit comprises the protein or polypeptide according to claim 9.

16. (canceled)

17. A kit for detecting an anti-citrullinated protein autoantibody, comprising the protein or polypeptide according to claim 19.

18. The method according to claim 1, wherein an integral structure of the cyclic peptide is KKCK-CCP linear amino acid sequence-DCDD.

19. The protein or polypeptide according to claim 7, wherein the protein is CCP linear amino acid, and the dimer zipper fastener parts are located at N-terminus and C-terminus of the CCP linear amino acid sequence, respectively.


SEQUENCE LISTING (4 sequences)

SEQ ID NO: 1 - protein, 107 aa, artificial sequence
  modified ESAT6 sequence
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGDDCDD

SEQ ID NO: 2 - protein, 102 aa, artificial sequence
  modified CFP10
MTEQQWNFAGIEAAASAIQGNVTSIHSLLDEGKQSLTKLAAAWGGSGSEAYQGVQQKWDATATELNNALQNLARTISEAGQAMASTEGNVTGMFAGGKKCKK

SEQ ID NO: 3 - protein, 10 aa, artificial sequence
  Flag-tag sequence
DYKDDDDKGG

SEQ ID NO: 4 - protein, 8 aa, artificial sequence
  His-tag sequence
HHHHHHGG